(19) United States
(12) Patent Application Publication
(10) Pub. No.: US 2010/0317891 A1
(43) Pub. Date: Dec. 16, 2010

(54) METHOD FOR THE PURIFICATION OF ORGANIC ACIDS

(75) Inventors: Marc-André Theoleyre, Paris (FR); Yvan Bathany, Vernouillet (FR)

Correspondence Address: ADELI & TOLLEN, LLP, 11940 San Vicente Blvd., Suite 100, LOS ANGELES, CA, 90049, US

(73) Assignee: APPLEXION, Epone (FR)

(21) Appl. No.: 12/521,566

(22) PCT Filed: Dec. 12, 2007
(86) PCT No.: PCT/FR2007/002150

§ 371 Date: Jun. 26, 2009

(30) Foreign Application Priority Data
Dec. 29, 2006 (FR) 0611545

(51) Int. Cl. C07C 51/42; C07C 51/43

(52) U.S. Cl. 562/513

(57) ABSTRACT

The invention relates to a method for the purification of worts containing optionally neutralised organic acids, which comprises the following steps: (a) removal of a portion at least of the divalent cations and optionally of a portion at least of the monovalent cations by passing the same on a cationic resin; and (b) nano-filtration of the resulting solution.

### FIELD OF THE INVENTION

A subject of the present invention is a method for the purification of organic acids, in particular originating from fermentation worts.

### STATE OF THE ART

Numerous organic acids, such as lactic, gluconic, citric, succinic and propionic acid, are produced in a standard fashion by fermentation from sugars, saccharose, glucose, lactose, etc. During the production of organic acid by fermentation, neutralization of the fermentation medium is necessary in order to avoid the inhibition of the fermentation by the acidity produced. In numerous cases, this neutralization is carried out by adding lime Ca(OH)₂ thus leading to the formation of calcium and organic acid. This neutralization can also be carried out by the addition of soda or ammonium hydroxide, leading to the formation of sodium and ammonium salts of the organic acid, respectively.

After fermentation, the first operation is clarification of the fermentation wort in order to eliminate the biomass from it. The subsequent purification means depend on the way in which the fermentation is carried out and in particular on the means utilized in order to control the pH during fermentation: lime, soda or ammonium hydroxide.

In the case of the method using lime, the fermentation medium is treated with sulphuric acid. Then, CaSO₄ (insoluble gypsum) is formed and organic acid is released in molecular form.

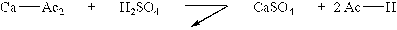

The gypsum thus formed is separated by filtration. The organic acid solution, saturated with CaSO₄ is then purified by conventional treatments of bleaching on activated carbon or resin, then demineralization on ion exchange resin or by a combination of electrodialysis and ion exchange.

In the methods using soda or ammonium hydroxide, after filtration the dissociation of the organic acid can be obtained by passing through cationic resin, regenerated with sulphuric or hydrochloric acid, or by bipolar membrane electrodialysis. The organic acid thus formed is then purified by conventional means.

WO-A-2004057008 describes the use of nanofiltration membranes in order to prepurify the wort after clarification. The main advantage of this nanofiltration technology is the effective elimination of the colourants. When glucose syrups are used, nanofiltration is also effective in eliminating the residual glucose polymers which are difficult to eliminate by other separation techniques. However, the implementation of this technology is limited by the calcium salts content of the fermentation worts and the risks of precipitation which are associated with this, due to their low solubility. These risks exist whatever the type of wort treated. The implementation of nanofiltration techniques must in fact be carried out under conditions for which there is no risk of precipitation of the mineral materials. In fact, such precipitation would lead to the irreversible clogging of the membranes. In most of the fermentation worts, the SO₄⁻ are the majority mineral anions, they have a tendency to form, with calcium, salts which are highly insoluble and particularly incrusting.

FR-A-2452879 describes a method for the preparation of dairy products comprising a decalcification stage which can be implemented before the ultrafiltration stage. This document relates to a technique in which the filtration does not have the risks associated with nanofiltration given the difference in pore size. The application WO-A-2004/022787 describes a method for the treatment of an aqueous solution containing sugars, comprising a stage (a) of replacement of the multivalent ions by monovalent ions, a nanofiltration stage (b) at the end of which a retentate and a permeate are recovered, and a stage (c) of complementary demineralization of the retentate in particular on resins, stage (b) being used here as a stage with a demineralization effect, since the sought product is the retentate and the monovalent ions pass through the nanofiltration membrane. In this patent application, demineralization by nanofiltration is sought.

A need still exists for a method for the treatment of worts using nanofiltration after clarification, without giving rise to the risks associated with the precipitation of calcium salts.

### SUMMARY OF THE INVENTION

The invention is based on an implementation under particular conditions allowing the use of nanofiltration membranes as a pretreatment, in particular pretreatment of the standard final treatment on resin and/or carbon. This pretreatment makes it possible to considerably reduce the load of organic polymers, minerals and colourants, by factors generally comprised between 1 and 3 with respect to minerals, and by a factor greater than 10 with respect to colourants. In the invention, the risks associated with the irreversible clogging of the membranes due to the precipitation of the mineral materials, in particular calcium salts, are avoided.

The invention is based on the combination of the previous method for elimination of the calcium on cationic resin before the nanofiltration. The invention, in an advantageous embodiment, also makes use of the secondary flows from the subsequent purification stages for the regeneration of the decalcification cationic resins.

The invention therefore provides a method for the purification of worts containing optionally neutralized organic acids, comprising the following stages:
(a) elimination of at least some of the divalent cations and optionally at least some of the monovalent cations by passing through a cationic resin; and
(b) nanofiltration of the solution resulting in a permeate.

According to an embodiment, the method according to the invention also comprises an acidification stage (ac) by contact with a cationic resin in the H⁺ form, which can be implemented before or after the nanofiltration stage (b).

According to an embodiment, in the method according to the invention, stage (a) is implemented by contact with a cationic resin in H⁺ form.

According to an embodiment, the method according to the invention also comprises the following stages:
(d1) treatment of the eluate from regeneration of the cationic resin of stage (a) and/or (ac) by bipolar membrane electrodialysis and production of an acid solution and a basic solution;
(e1) regeneration at least in part of the resin of stage (a) using the acid solution of stage (d1).

According to an embodiment, the method according to the invention also comprises the stage of neutralization of the wort, during fermentation, at least in part using the basic solution of stage (d1).

According to an embodiment, in the method according to the invention stage (a) is implemented by contact with a cationic resin in Na⁺ and/or K⁺ form.

According to an embodiment, the method according to the invention also comprises the following stages:
(d2) treatment of the eluate from regeneration of the cationic resin of stage (a) by nanofiltration and production of a saline solution in the retentate;
(e2) regeneration at least in part of the resin of stage (a) using the saline solution of stage (d2).

According to an embodiment, the method according to the invention also comprises the following stage:
(c) purification of the permeate of stage (b), preferably by a technique chosen from the group consisting of demineralization, crystallization, chromatography, electrodialysis, and combinations thereof.

According to an embodiment, in the method according to the invention stage (c) is a demineralization stage, preferably implemented on exchange resins.

According to an embodiment, the method according to the invention also comprises the following stages:
(f) treatment of the eluates from regeneration of the demineralization resins of stage (c) by nanofiltration and production of a saline solution in the retentate;
(e) regeneration at least in part of the resin of stage (a) using the saline solution of stage (f).

According to an embodiment, in the method according to the invention stages (d2) and (f) are implemented in combination with each other.

According to an embodiment, the method according to the invention also comprises the following stages:
(g) combination of the nanofiltration retentate from stage (b) with the nanofiltration retentate from stage (d2);
(h) precipitation of CaSO₄ and production in the supernatant of a saline solution;
(e) regeneration at least in part of the resin from stage (a) using the saline solution from stage (h).

According to an embodiment, the method according to the invention also comprises the following stage:
(i) concentration of the effluent originating from purification stage (c).

According to an embodiment, in the method according to the invention the acid solution is a clarification solution of fermentation worts.

According to an embodiment, in the method according to the invention, the acid is a diacid.

According to an embodiment, in the method according to the invention the acid is chosen from the group consisting of lactic, gluconic, citric, succinic, propionic acid, and mixtures thereof.

### BRIEF DESCRIPTION OF THE FIGURES

FIG. 1 diagrammatically represents the method according to the invention;

FIG. 2 diagrammatically represents an embodiment of the method according to the invention;

FIG. 3 diagrammatically represents another embodiment of the method according to the invention;

### DETAILED DESCRIPTION OF EMBODIMENTS OF THE INVENTION

The invention applies generally to all the organic acids resulting from fermentation; there can be mentioned lactic, gluconic, citric, succinic, propionic acids, etc. The invention also applies to the various neutralization methods, such as methods using lime, soda, ammonium hydroxide, in particular lime.

The worts treated in the invention originate from the standard clarification stage which makes it possible to separate the biomass from the acid effluents produced.

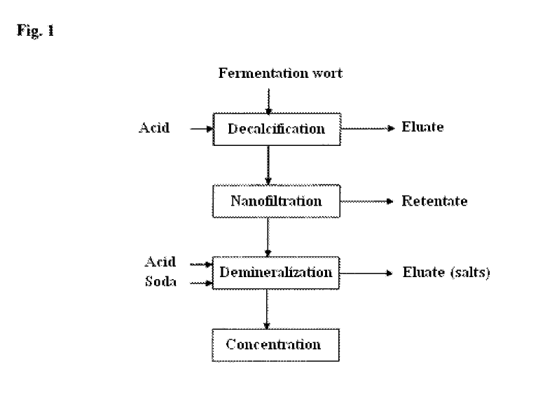

With reference to FIG. 1, the invention uses an acidified or non-acidified wort. The pH of the wort can be comprised between 1.5 and 5.5 depending on the pKa of the organic acid considered.

This wort is subjected, in a first phase, to decalcification on a cationic resin. This cationic resin can be of the H⁺ type, or of the Na⁺ or K⁺ type. The cationic resin is regenerated for example by an acid in the case where the cationic resin is of the H⁺ type, as represented in FIG. 1. Examples of resins are XA2023 or XA2033 from APPLEXION.

In the case where an H⁺ form resin is used, the treatment on resin can be carried out so as to ensure two functions: elimination of the calcium ions, and hydrolysis of the organic acid salt to its acid form by binding of all the mineral cations including those bound to the organic acid. These two functions can be performed by a single operation on a single H⁺ resin or two operations in series. In the latter case the first resin is dimensioned such that saturation of the exchange sites occurs substantially after exchange of the calcium ions; the outward flow then being an organic acid salt free from the calcium which could optionally be present. When hydrolysis of the salt is sought, a continuous ion exchange treatment is particularly suitable. This solution is preferred in the case of diacids (for example succinic acid) for which the salt form has a retention greater than that of the acid form.

The acidification stage can be implemented before or after the nanofiltration, and preferably before the nanofiltration stage in the case of the organic diacids.

In the case of the H⁺ form resins, regeneration of the resin is carried out by passage of sulphuric or hydrochloric acid and the eluates constitute a solution of mostly ammonium or sodium salts (in particular ammonium or sodium sulphate) depending on whether the pH of the fermentation has been controlled with ammonia or soda. In the case of a solution containing calcium ions, it is possible to carry out a first treatment of decalcification on resin, so as to remove the calcium ions. It is possible to implement a bipolar membrane electrodialysis technique on the eluate (when the acid salt contains calcium, a stage of suppression of the calcium is implemented before the acidification on H⁺ resin such that the eluate is low in calcium). This makes it possible to produce on the one hand a basic ammonia or soda solution which can be used for controlling fermentation pH and on the other hand an acid solution which can be reused for the regeneration of the resins. The use of concentrated solutions is favoured in this case, for the bipolar membrane electrodialysis, as the surface area of the membranes is reduced and the conductivity is improved.

During the first stage, in general at least 60%, preferably at least 80%, advantageously at least 90% or even at least 95% of the divalent cations (calcium) are removed. If an Na⁺ or K⁺ type resin is used, the monovalent ions of course remain in the solution. If an H⁺ type resin is used, depending on the dimensioning, it is possible to choose to eliminate only the divalent cations or, by contrast, to carry out complete acidification and also eliminate the monovalent cations. The elimination of the monovalent cations can therefore be comprised between 0% and at least 90%, depending on the case. In general, either the monovalent ions are not substantially removed, or they are substantially removed (at least 90%, or even at least 95%).

After decalcification, the majority of the sulphate ions are to be found in solution in the form of sulphuric, hydrogen sulphuric acid or in the form of the sulphate of monovalent cations, sodium or potassium, highly soluble and easily retained by nanofiltration membranes. The invention therefore allows the use of the nanofiltration membranes without fear of blockage by precipitation of calcium salts.

This solution is therefore then treated by nanofiltration on PERSEP 100 or PERSEP 200 type membrane from APPLEXION. The permeate is recovered and it is then sent to a standard purification stage, for example demineralization.

This demineralization stage is implemented in a standard manner, for example a method by electrodialysis or resins or a combination of the two. The effluent from this stage, which is rich in monovalent ions, can then be used for regeneration of the decalcification resin, when the resin is used in the Na⁺ and/or K⁺ form. In certain cases, a complementary treatment of bleaching on carbon can also be implemented.

The final stage is, in a standard manner, a stage of concentration of the acid solution, which can be implemented by standard techniques such as reverse osmosis and/or evaporation.

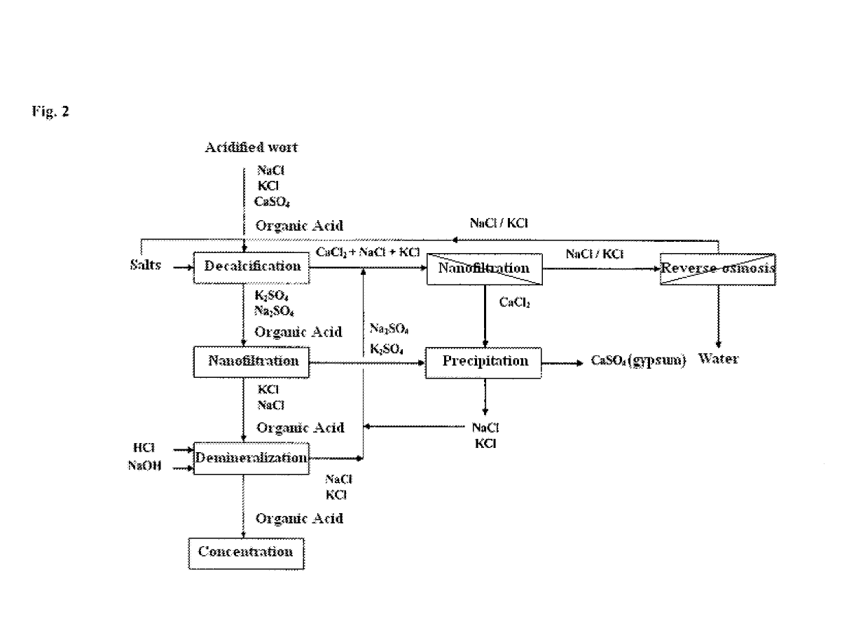

With reference to FIG. 2, the invention uses a treatment which makes use of secondary flows for the regeneration of the resins used in the invention. The acidified wort (treated for example with sulphuric acid) is shown at the top of the method, and the majority species, NaCl, KCl, CaSO₄, and the sought organic acid are indicated. In a first stage this acidified wort undergoes decalcification on a strong cationic resin in the monovalent form, for example Na⁺ and/or K⁺, regenerated by a salt solution: NaCl and/or KCl.

The flow leaving the decalcification stage this time contains as majority species the acid and the majority of the sulphate ions in the form of sodium or potassium sulphate.

The eluate from regeneration of the decalcification resin, rich in highly soluble calcium chloride, can also be treated by nanofiltration in order to concentrate the CaCl₂ in a retentate and to recover an almost pure solution of sodium and potassium chloride in the permeate, which can be used to regenerate the decalcification resins. The regeneration of the decalcification resin is represented by the loop towards the decalcification resin which makes use of NaCl/KCl salts. It is possible to provide additional stages of concentration and/or reverse osmosis, in particular after the stage of nanofiltration of the cationic resin regeneration eluate.

The flow originating from the decalcification is sent to a nanofiltration stage which makes it possible to separate the multivalent salts and the glucose polymers as well as the colourants. At the nanofiltration stage, it is thus possible to eliminate, apart from the colourants and apart from the macromolecules, the majority of the ions. Overall, elimination of 40 to 65% of cations and 50 to 75% of the mineral anions is observed.

The diafiltration treatment (not shown) of the retentate makes it possible to recover the lactic acid that it contains and thus improve the overall yield of the method.

The nanofiltration permeate is then sent to a complementary purification stage. This stage can be a standard demineralization stage, in particular based on the use of two resins (cationic and anionic), as shown in FIG. 2. It is also possible to use crystallization, chromatography, electrodialysis, etc.

For the final regeneration of the demineralization resins, hydrochloric acid and soda are preferably used. Thus, after mixing, the regeneration eluates are mostly constituted by NaCl salts capable of being used for the regeneration of the decalcification resin, optionally after nanofiltration treatment, concentration and/or reverse osmosis.

The retentate of this nanofiltration of the eluates, enriched with CaCl₂, is mixed with the nanofiltration retentate of the product enriched with NaSO₄ in order to eliminate the sulphates in the form of CaSO₄ by precipitation. A solution of monovalent salts (NaCl) is then available which can be used for the regeneration of the resins.

Thus, the recycling of diluted fractions rich in monovalent salts is sufficient to regenerate the decalcification resins which do not then require any input of chemical product for their regeneration. It is thus possible to optimize the flows in the method.

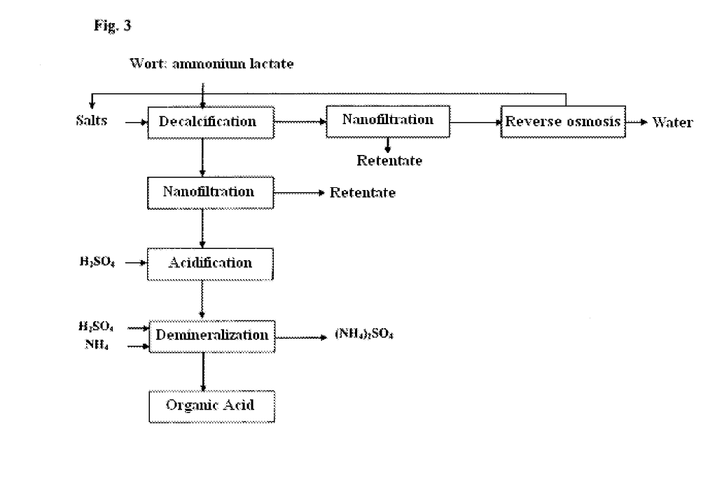

With reference to FIG. 3, the invention treats a wort from a neutralized solution. The raw material treated can be ammonium hydroxide (or soda) depending on the method. The organic acid is in this case in neutralized form, the pH can in particular be comprised between 3 and 10. In this case, for example the major part of the lactic acid is in the form of ammonium lactate. Passing through a strong cationic resin allows the elimination of the calcium salts present in the medium, in a manner identical to the embodiments of FIGS. 1 and 2.

Treatment by nanofiltration makes it possible to eliminate the colourants, macromolecules, proteins and glucose polymers, but also, like in the embodiments of FIGS. 1 and 2, the sulphates present in the form of sodium, potassium or ammonium sulphate.

The permeate rich in lactate, for example ammonium or sodium lactate, is then acidified, for example on a strong H⁺ form cationic resin, regenerated with sulphuric acid or hydrochloric acid, at the output of which a molecular lactic acid solution is recovered.

It is also possible to use a continuous ion exchange method which makes it possible to improve the load on the resin while reducing the consumption of water and reagents for the regeneration.

If necessary, the ammonium sulphate optionally produced during the regeneration is separated into sulphuric acid and ammonia, for example by bipolar membrane electrodialysis. This acidification on resins can be done before the nanofiltration, in particular in the case of divalent organic acids (for example succinic). As mentioned above, it is possible to use two resins in series.

An alternative to this acidification treatment on cationic resin is a bipolar membrane electrodialysis making it possible to produce a flow of lactic acid and a flow of ammonium hydroxide.

The total demineralization of the acid is obtained by passing in series through cationic and anionic finishing resins, like in the embodiments of FIGS. 1 and 2.

Generally, the method according to the invention is implemented at a temperature comprised between 20 and 60° C.

The invention applies particularly to the solutions for clarification of fermentation worts, in particular clarification of the fermentation worts according to reverse osmosis and/or evaporation techniques.

The following examples illustrate the invention without limiting it.

### EXAMPLES

### Example 1

The fermentation wort, originating from a so-called lime method is acidified by treatment with sulphuric acid, the gypsum thus formed is eliminated by filtration. The aqueous medium then contains calcium sulphate, which represents the major part of the ions present in the filtrate. This solution is treated on a strong XA 2033, XA 2023 type cationic resin from APPLEXION, regenerated with hydrochloric acid, in order to eliminate first and foremost the multivalent cations (but also some of the monovalent cations). The resulting solution then contains species which result from the ion exchange. The solution is then treated by nanofiltration on Persep 100 or 200 membrane from APPLEXION. The permeate is then demineralized by ion exchange (XA2023 and XA3061 from APPLEXION) then concentrated by evaporation.

During the nanofiltration stage, the multivalent anions SO₄ are mostly concentrated in the retentate as well as the glucose polymers and the macromolecules. At this stage, in particular, effective elimination of the colourants is noted. The permeate and the retentate contain lactic acid (the lactic acid passes directly into the permeate and therefore there is no substantial concentration), whereas a concentration of ions is noted. This permeate can then be purified by the conventional techniques.

The results are given in the table below (in which CFV is the Concentration Factor by Volume=initial volume/volume of retentate; and in which OD is the Optical Density (colour measured by the optical density at 420 nm), “cat” means mineral cation, “an” means mineral anion, “div” means divalent, “monov” means “monovalent”, “lact ac” means lactic acid).

Example 1: Treatment of the wort by nanofiltration at CFV 10

| | | lact | div | monov | div | monov | |
|---|---|---|---|---|---|---|---|
| | | ac | cat | cat | an | an | |
| | vol l | g/l | meq/l | meq/l | meq/l | meq/l | OD |
| Acidified wort | | 100 | 30.0 | 8.0 | 31.0 | 1.3 | |
| Decalcified wort | 100 | 100 | 3.0 | 0.8 | 31.0 | 1.3 | 0.25 |
| NF retentate | 10 | 127 | 19.0 | 4.0 | 166.0 | 7.0 | 2.16 |
| NF permeate | 90 | 97 | 1.2 | 0.4 | 16.0 | 0.7 | 0.04 |

### Example 2

This example is implemented on an installation as described in FIG. 2. In this example, the lactic fermentation wort, after treatment with sulphuric acid, is passed through a strong XA 2023 type monovalent form cationic resin, from APPLEXION. After decalcification, the majority of the sulphate ions is found in solution in the form of sodium or potassium sulphate, highly soluble and easily retained by nanofiltration membranes. The separation takes place as in Example 1, the sulphate ions being in the retentate. The diafiltration treatment of the retentate (before precipitation) makes it possible to recover the lactic acid that it contains and thus improve the overall yield of the method. The permeate is then demineralized as in Example 1.

The eluate from regeneration of the decalcification resin, which is rich in highly soluble calcium chloride, is treated by nanofiltration in order to concentrate the CaCl₂ in a retentate and to recover in the permeate an almost pure sodium and potassium chloride solution, which is used to regenerate the decalcification resins.

The permeate of this nanofiltration of the eluates, enriched with CaCl₂ is then mixed with the nanofiltration retentate of the product enriched with Na₂SO₄ in order to eliminate the sulphates in the form of CaSO₄ by precipitation. An NaCl solution is then available which is used for the regeneration of the resins.

For the final regeneration of the demineralization resins of the main flow, the nanofiltration permeate, hydrochloric acid and soda are used. Thus, after mixing, the regeneration eluates are mostly constituted by NaCl salts which after treatment by nanofiltration, concentration by reverse osmosis, are used for the regeneration of decalcification resins, as indicated in FIG. 2.

The results are given in Table 1 below.

Example 2: Treatment of the wort by nanofiltration at CFV 15

| | | lact | div | monov | div | monov | |
|---|---|---|---|---|---|---|---|
| | | ac | cat | cat | an | an | |
| | vol l | g/l | meq/l | meq/l | meq/l | meq/l | OD |
| Acidified wort | | 100 | 30 | 8 | 30 | 1.3 | |
| Decalcified wort | 100.0 | 100 | 0.3 | 42 | 30 | 1.3 | 0.24 |
| NF retentate | 6.7 | 133 | 2.6 | 327 | 366 | 9.6 | 3.15 |
| NF permeate | 94.3 | 97.6 | 0.1 | 21.6 | 6 | 0.7 | 0.03 |

### Example 3

In this example, the raw material is a fermentation wort according to the ammonium hydroxide method; the organic acid is in this case in neutralized form. In this case, the major part of the lactic acid is in the form of ammonium lactate. Passing through strong H⁺ cationic resin allows the elimination of the calcium salts present in the medium, in a manner similar to Examples 1 and 2. This resin is dimensioned in order substantially to exchange only the divalent ions (an acidification may optionally appear, but without complete hydrolysis). The nanofiltration treatment makes it possible to eliminate the colourants, macromolecules, proteins and glucose polymers, but also, as in Examples 1 and 2, the sulphates present in the form of sodium, potassium or ammonium sulphate.

The permeate rich in ammonium lactate is then treated for hydrolysis and acidification on a strong H⁺ form cationic resin, regenerated with sulphuric acid after which on a solution of molecular lactic acid is recovered. The regeneration with sulphuric acid leads to the formation of ammonium sulphate.

It is also possible to acidify the wort before the nanofiltration stage, in a general manner.

The total demineralization of the acid is obtained by passing in series through cationic and anionic finishing resins, as in Examples 1 and 2.

The results are given in Table 1 below.

Example 3: Treatment of the wort by nanofiltration at CFV 10

| | | lact | div | monov | div | monov | |
|---|---|---|---|---|---|---|---|
| | | ac | cat | cat | an | an | |
| | vol l | g/l | meq/l | meq/l | meq/l | meq/l | OD |
| Acidified wort | | 100 | 19 | 1060 | 9.5 | 1.3 | |
| Decalcified wort | 100 | 100 | 0.2 | 1079 | 9.5 | 1.3 | 0.24 |
| NF retentate | 10 | 127 | 1.2 | 2330 | 90 | 5.6 | 2.17 |
| NF permeate | 90 | 97 | 0.1 | 940 | 0.5 | 0.9 | 0.02 |

1. A method for the purification of worts containing optionally neutralized organic acids, comprising the following stages:
(a) elimination of at least part of the divalent cations and optionally at least part of the monovalent cations by passing through a cationic resin; and
(b) nanofiltration of the resulting solution.

2. The method of claim 1, wherein the method also comprises an acidification stage (ac) by contact with a cationic resin in the H⁺ form, which can be implemented before or after the nanofiltration stage (b).

3. The method of claim 1, wherein stage (a) is implemented by contact with a cationic resin in the H⁺ form.

4. The method of claim 1, wherein the method also comprises the following stages:
(d1) treatment of the eluate from regeneration of the cationic resin of stage (a) and/or (ac) by bipolar membrane electrodialysis and production of an acid solution and a basic solution;
(e1) regeneration at least in part of the resin of stage (a) using the acid solution of stage (d1).

5. The method of claim 4, wherein the method also comprises the stage of neutralization of the wort, during fermentation, at least in part using the basic solution of stage (d1).

6. The method of claim 1, wherein stage (a) is implemented by contact with a cationic resin in the Na⁺ and/or K⁺ form.

7. The method of claim 1, wherein the method comprises the following stages:
(d2) treatment of the eluate from regeneration of the cationic resin of stage (a) by nanofiltration and production of a saline solution in the retentate;
(e2) regeneration at least in part of the resin of stage (a) using the saline solution of stage (d2).

8-16. (canceled)

17. The method of claim 1, wherein the acid is a diacid.

18. The method of claim 1, wherein the acid is chosen from the group consisting of lactic, gluconic, citric, succinic, propionic acid, and mixtures thereof.

19. The method of claim 7, wherein the method also comprises the following stages:
(g) combination of the nanofiltration retentate from stage (b) with the nanofiltration retentate from stage (d2);
(h) precipitation of CaSO₄ and production in the supernatant of a saline solution;
(e) regeneration at least in part of the resin from stage (a) using the saline solution from stage (h).

20. The method of claim 1, wherein the method also comprises the following stage:
(c) purification of the permeate of stage (b).

21. The method of claim 20, wherein the method also comprises the following stage:
(i) concentration of the effluent originating from the purification stage (c).

22. The method of claim 20, wherein stage (c) is a demineralization stage.

23. The method of claim 22, wherein the method also comprises the following stages:
(f) treatment of the eluates from regeneration of the demineralization resins of stage (c) by nanofiltration and production of a saline solution in the retentate;
(e) regeneration at least in part of the resin of stage (a) using the saline solution of stage (f).

24. The method of claim 23, wherein the stages (d2) and (f) are implemented in combination with each other.

25. The method of claim 20, wherein stage (c) is a demineralization stage implemented on exchange resins.

26. The method of claim 1, wherein the purification of the permeate of stage (b) is performed by a technique chosen from the group consisting of demineralization, crystallization, chromatography, electrodialysis, and combinations thereof.

27. The method of claim 1, wherein the acid solution is a clarification solution of fermentation worts.
